(19) United States
(12) Patent Application Publication
(10) Pub. No.: US 2003/0044372 A1
(43) Pub. Date: Mar. 6, 2003

(54) Stable aqueous emulsions of alkoxytrimethylsilane fluids

(76) Inventor: Gary E. LeGrow, Newberry, FL (US)

Correspondence Address: Alan B. Clement, Esq., HEDMAN & COSTIGAN, P.C., Suite 2003, 1185 Avenue of the Americas, New York, NY, 10036-2646, US

(21) Appl. No.: 09/942,483

(22) Filed: Aug. 30, 2001

(51) Int. Cl. A61K 7/06; A61K 7/11; C09K 3/00

(52) U.S. Cl. 424/70.12; 516/55

(57) ABSTRACT

The present invention relates to stable aqueous emulsions of liquid alkoxytrimethylsilanes comprising a continuous aqueous phase, a discontinuous organic and/or alkoxytrimethylsilane phase and an emulsification system. The emulsification system comprises two emulsifying agents, an organosilicone hydrophobic emulsifying agent and an organosilicone hydrophilic emulsifying agent. Emulsions of this type are especially useful for delivery of liquid alkoxytrimethylsilanes to the surface and upper layers of the stratum corneum. A method for preparing these emulsions is also disclosed.

### FIELD OF THE INVENTION

The present invention relates to aqueous emulsions comprising an alkoxytrimethylsilane that can be used for delivering alkoxytrimethylsilanes to surfaces on the human body, including, but not limited to, the stratum corneum and the hair. More particularly, this invention relates to aqueous emulsions comprising an alkoxytrimethylsilane that remains intact with time under normal ambient conditions with no measurable change in the discontinuous phase, no particle aggregation, and no chemical transformations.

### BACKGROUND OF THE INVENTION

A wide range of long chain aliphatic alcohols are used in cosmetic formulations, including skin care and hair care products, because of some of the inherently desirable characteristics of these materials including occlusivity, substantivity, water repellency, and ultraviolet radiation protection. Long chain aliphatic alcohols, possessing these characteristics, typically have carbon chain lengths ranging from 12 upwards to more than 20 carbon atoms. When pure, all of these alcohols are solids at room temperature. In formulating cosmetic products, higher melting ingredients are frequently melted, such as in a hot room, and then formulated while in the molten state. The requirement of performing this melting step represents a significant disadvantage to the use of solid ingredients in formulating cosmetic products.

It therefore would represent a significant advance in the state of the art if a composition that is useful in providing the beneficial characteristics of solid long chain aliphatic alcohols could be developed that does not also possess the drawback of requiring melting prior to formulation. In this regard, mention is made here of U.S. Pat. No. 5,847,179, which discloses liquid alkoxytrimethylsilanes, trimethylsilyl derivatives of solid long chain aliphatic alcohols, that can be formulated readily into cosmetic products at room temperature. An even greater advance in the state of the art of cosmetic formulations, however, would be the ability to formulate these liquid long chain aliphatic alkoxytrimethylsilanes under ambient conditions into aqueous emulsions and retain the liquid alkoxytrimethylsilane structure intact.

Mention is also made of Fisher, U.S. Pat. No. 6,074,470 which teaches a non-silicone emulsifying system consisting essentially of a non-ionic primary surfactant having an HLB greater than 13 and a secondary non-ionic co-surfactant having an HLB less than 11 for emulsifying alkoxysilanes useful as water repellents.

SUMMARY OF THE INVENTION

The present invention meets the problems of the prior art and provides stable aqueous emulsions comprising fluid alkoxytrimethylsilanes in the discontinuous oily phase and a method for their preparation. The aqueous emulsions comprise an alkoxytrimethylsilane, an organosilicone hydrophobic emulsifier, an organosilicone hydrophilic emulsifier, and water. The aqueous emulsions exhibit improved stability in that the discontinuous particles do not increase in size with age, over at least a period of three months, and there is no apparent indication of hydrolysis of the alkoxytrimethylsilane.

### DETAILED DESCRIPTION OF THE INVENTION

The aqueous emulsions of the present invention are stable and do not phase separate. They have constant particle size over at least a three-month period under ambient conditions. In the case of fluid alkoxytrimethylsilanes derived from solid alcohols, another measure of stability of the aqueous emulsion is the absence of solid alcohol over time, the presence of which would be accounted for by hydrolysis of the alkoxytrimethylsilane.

The present invention provides an aqueous liquid alkoxytrimethylsilane containing emulsion comprising:

(a) a continuous phase comprising water;

(b) a discontinuous phase comprising at least one liquid alkoxytrimethylsilane, wherein the discontinuous phase forms particles which are uniformly dispersed in the continuous phase; and

(c) a dual emulsifier system consisting essentially of an organosilicone hydrophobic surfactant and an organosilicone hydrophilic surfactant.

The organosilicone hydrophobic emulsifying agent is believed, although the inventor does not wish to be bound to any theory, to prevent diffusion of the liquid organic and/or alkoxytrimethylsilane particle into the continuous aqueous phase. The organosilicone hydrophilic emulsifying agent is believed, although again the inventor does not wish to be bound to any theory, to prevent coalescence of the discontinuous organic and/or alkoxytrimethylsilane particles.

The alcohol, from which the liquid alkoxytrimethylsilane is formed, has an aliphatic hydrocarbon structure with from twelve to more than 22 carbon atoms which may be acyclic, cyclic or both, and which may contain unsaturated groups.

The amount of liquid alkoxytrimethylsilane in the discontinuous phase of the aqueous emulsion may range from 1 to 100 percent. In addition to the alkoxytrimethylsilane, the present invention contemplates the inclusion of inert organic diluents such as, for example but not limited to, soybean oil, mineral oil or other vegetable oils, or organic esters such as isopropylmyristate, and the like, or mixtures thereof, in the discontinuous phase of the emulsion. The amount of the discontinuous phase in the aqueous emulsion may range from about 5 to about 50 percent.

Liquid alkoxytrimethylsilanes, useful in the aqueous emulsions of the present invention and in cosmetic applications, include, but are not limited to, those containing alkoxy groups with aliphatic hydrocarbon structures with from 12 to more than 22 carbon atoms, which may be cyclic, acyclic or both, and which may contain unsaturated groups. Suitable examples include retinoxytrimethylsilane, stearoxytrimethylsilane, cetoxytrimethylsilane, cetearoxytrimethylsilane, arachidoxytrimethylsilane, behneoxytrimethylsilane, palmitoleooxytrimethylsilane, oleoxytrimethylsilane, linoleoxytrimethylsilane, linolenoxytrimethylsilane, arachidonoxytrimethylsilane, erucoxytrimethylsilane and mixtures of any of the foregoing. In preferred embodiments of the present invention, the alkoxytrimethylsilane fluids can be prepared according to the methods disclosed in LeGrow et al., U.S. Pat. No. 5,847,179.

Organosilicone hydrophobic surfactants suitable for use with the aqueous emulsions of the present invention are of the alkyl-silicone-polyoxyalkylene type. Alkysilicone-polyoxyalkylene copolymers are particularly suited for encapsulation of hydrocarbons and/or silanes in aqueous emulsions because of their affinity for both hydrocarbon and silane compositions. The concentration of the organosilicone hydrophobic surfactant in the emulsion should be sufficient to continuously cover the surface of the discontinuous particles, typically one to three molecules per 100 Angstroms of surface area.

Organosilicone hydrophobic surfactants of the alkyl-silicone-polyoxyalkylene type, which are suitable for use in the practice of the present invention and which are commercially available include, but are not limited to, DC-5200 which is Lauryl PEG/PPG-18/18 methicone copolymer, produced by Dow Corning Corporation; Abil® EM-90 which is Cetyl PEG/PPG-10/1 dimethicone copolymer, produced by Goldschmidt TM AG and SilCare™ 140M30 Caprylyl Bis-(PEG/PPG-20/20) dimethicone copolymer, produced by Clariant. This latter product is an example of a class of surfactants disclosed in currently pending, commonly assigned, U.S. patent application Ser. No. 09/549,325 filed on Nov. 13, 2000 (attorney docket 577-124) by the same inventor.

Organosilicone hydrophilic surfactants suitable in these emulsions are of the water-soluble silicone-polyoxyethylene type, of the general formula:

(Me₃SiO)₂MeSi(CH₂)₃O(CH₂CH₂O)ₓMe

wherein x may range from 6 to about 20. Water-soluble silicone-polyoxyethylene copolymers are particularly suited to prevent coalescence of the discontinuous organic and/or alkoxytrimethylsilane particles because of their affinity for the alkoxytrimethylsilane and their ability to reduce the surface tension of water in their environment. The concentration of the hydrophilic surfactant in the emulsions of the present invention should be sufficient to cover the surface of the hydrophobic surfactant treated discontinuous particles, typically two to six molecules per 100 square Angstroms of surface area. An organosilicone hydrophilic surfactant, which is suitable for use with the aqueous emulsions of the present invention, and which is commercially available is, but is not limited to, SilCare™ 140M40 Trisiloxane PEG-10 copolymer produced by Clariant. Reference is also made to Hill, Randall M., ed., “Silicone Surfactants,” Marcel Dekker Surfactant Science Series, vol. 86, 1999, pp. 241-48 which although related to herbicide formulations provides a further description of these materials and commercially available examples that are useful in the practice of the present invention.

The aqueous emulsions may further comprise one or more of the following components:

(d) a biocide;

(e) a buffering agent;

(f) a fragrance;

(g) a colorant;

(h) a foaming agent;

(i) an anti-foaming agent; and/or

(j) a thickener.

The aqueous emulsions of the present invention may be used in cosmetic formulations for delivery of the liquid alkoxytrimethylsilane, which they contain, to the surface of the skin or hair. The spreadability and penetrating characteristics of liquid alkoxytrimethylsilanes, as is well known to those skilled in the art, then provide more efficient delivery to the skin or hair, thereby making these emulsions more efficacious. Once in the upper layers of the stratum corneum or on the hair, these emulsions break liberating liquid alkoxytrimethylsilanes, which are then hydrolysed by contact with water liberating the parent solid alcohols.

### DESCRIPTION OF THE PREFERRED EMBODIMENTS

The following examples are presented to illustrate the present invention. These examples are not to be construed to limit the scope of the appended claims in any manner whatsoever.

### COMPARATIVE EXAMPLE 1

An aqueous emulsion of liquid stearoxytrimethylsilane was prepared by the following method. Forty grams of stearoxytrimethylsilane were mixed with 1.4 grams (0.117 mmole) of SilCare™ Caprylyl Bis(PEG/PPG-20/20) dimethicone copolymer forming a hazy solution (I). Over a period of 10 minutes, solution (I) was slowly added to twelve grams of water using a Tissue-Tearor homogenizer running in the range of 4000-8000 rpm. After complete addition, the homogenizer was run for an additional 20 minutes. To this mixture was added 46.6 grams of de-ionized water, using the homogenizer, over a period of 10 minutes. After complete addition, the homogenizer was run for an additional 20 minutes. Finally, 0.1 gram of sodium bicarbonate was added to the mixture and blended with the homogenizer for 10 minutes.

A sample of the above aqueous emulsion was examined with a Fisher optical microscope using 1000× magnification (1 unit on the scale=1 micrometer). The discontinuous “oil-phase” particles were observed to be of varying size, ranging from about 200 to about 500 nanometers in diameter. Within 12 hours, this sample showed a large phase separation and solid crystalline stearyl alcohol started to form. Within 24 hours the sample thickened significantly.

### EXAMPLE 1

An aqueous emulsion of liquid stearoxytrimethylsilane was prepared by the following method. Twelve (12) grams of water were mixed with 0.5 grams (0.665 mmole) of SilCare™ 140M40 Trisiloxane PEG-10 copolymer forming a clear solution (I). Forty grams of stearoxytrimethylsilane were mixed with 2.8 (0.272 mmole) of SilCare™ 14M30 Caprylyl Bis(PEG/PPG-20/20) dimethicone copolymer until a hazy mixture was formed (II). Over a period of 10 minutes, mixture (II) was slowly added to solution (I) using a Tissue-Tearor homogenizer running in the range of 4000-8000 rpm. After complete addition, the homogenizer was run for an additional 20 minutes. To this mixture was added 44.7 grams of deionized water, using the homogenizer, over a period of 10 minutes. After complete addition, the homogenizer was run for an additional 20 minutes. Finally, 0.1 gram of sodium bicarbonate was added to the mixture and blended with the homogenizer for 10 minutes.

A sample of the above aqueous emulsion was examined with the Fisher optical microscope as described in Comparative Example 1. The discontinuous “oil-phase” particles were observed to be of uniform size and approximately 250 nanometers in diameter. Samples of this emulsion were periodically inspected microscopically over the succeeding 3 months and were found to retain their particle size.

### EXAMPLE 2

An aqueous emulsion of liquid stearoxytrimethylsilane was prepared by the following method. Twelve (12) grams of water were mixed with 1.0 gram (1.330 mmole) of SilCare™ 140M40 Trisiloxane PEG-10 copolymer forming a clear solution (I). Forty grams of stearoxytrimethylsilane were mixed with 2.8 grams (0.272 mmole) of SilCare™ 140M30 Caprylyl bis(PEG/PPG-20/20) dimethicone copolymer until a hazy mixture was formed (II). Over a period of 10 minutes, mixture (II) was slowly added to solution (I) using a Tissue-Tearor homogenizer running in the range of 4000-8000 rpm. After complete addition, the homogenizer was run for an additional 20 minutes. To this mixture was added 45.1 grams of deionized water, using the homogenizer, over a period of 10 minutes. After complete addition, the homogenizer was run for an additional 20 minutes. Finally, 0.1 gram of sodium bicarbonate was added to the mixture and blended with the homogenizer for 10 minutes.

A sample of the above aqueous emulsion was examined with the Fisher optical microscope described in Comparative Example 1. The discontinuous “oil-phase” particles were observed to be of uniform size and approximately 250 nanometers in diameter. Samples of this emulsion were periodically inspected microscopically over the succeeding 3 months and were found to retain their particle size.

### EXAMPLE 3

An aqueous emulsion of liquid stearoxytrimethylsilane was prepared by the following method. Twelve (12) grams of water were mixed with 1.0 gram (1.330 mmole) of SilCare™ 140M40 Trisiloxane PEG-10 copolymer forming a clear solution (I). Forty grams of stearoxytrimethylsilane were mixed with 1.4 grams (0.136 mmole) of SilCare™ 140M30 Caprylyl bis(PEG/PPG-20/20) dimethicone copolymer until a hazy mixture was formed (II). Over a period of 10 minutes, mixture (II) was slowly added to solution (I) using a Tissue-Tearor homogenizer running in the range of 4000-8000 rpm. After complete addition, the homogenizer was run for an additional 20 minutes. To this mixture was added 45.6 grams of deionized water, using the homogenizer, over a period of 10 minutes. After complete addition, the homogenizer was run for an additional 20 minutes. Finally, 0.1 gram of sodium bicarbonate was added to the mixture and blended with the homogenizer for 10 minutes.

A sample of the above aqueous emulsion was examined with the Fisher optical microscope described in Comparative Example 1. The discontinuous “oil-phase” particles were observed to be of uniform size and approximately 250 nanometers in diameter. Samples of this emulsion were periodically inspected microscopically over the succeeding 3 months to retain their particle size.

### EXAMPLE 4

An aqueous emulsion of liquid stearoxytrimethylsilane in soybean oil was prepared by the following method. Twelve (12) grams of water were mixed with 0.5 grams (0.665 mmole) of SilCare™ TM 140M40 Trisiloxane PEG-10 copolymer forming a clear solution (I). A solution of 5.0 grams of stearoxytrimethylsilane in 35.0 Grams of soybean oil was mixed with 2.8 grams (0.272 mmole) of SilCare™ 140M30 Caprylyl bis(PEG/PPG-20/20) dimethicone copolymer forming a hazy mixture (II). Over a period of 10 minutes, mixture (II) was slowly added to solution (I) using a Tissue-Tearor homogenizer running in the range of 4000-8000 rpm. After complete addition, the homogenizer was run for an additional 20 minutes. To this mixture was added 44.7 grams of deionized water, using the homogenizer, over a period of 10 minutes. After complete addition, the homogenizer was run for an additional 20 minutes. Finally, 0.1 gram of sodium bicarbonate was added to the mixture and blended with the homogenizer for 10 minutes.

A sample of the above aqueous emulsion was examined with the Fisher optical microscope described in Comparative Example 1. The discontinuous “oil-phase” particles were observed to be of uniform size and approximately 250 nanometers in diameter. Samples of this emulsion were periodically inspected microscopically over the succeeding 3 months to retain their particle size.

The above-mentioned patents, patent applications and publications are hereby incorporated by reference.

Many variations of the invention will suggest themselves to those skilled in the art in light of the above-detailed description. All such obvious variations are within the full intended scope of the appended claims.

1. An aqueous emulsion comprising:
a) a continuous phase comprising water;
b) a discontinuous phase comprising at least one liquid alkoxytrimethylsiliane, wherein the discontinuous phase forms particles which are uniformly dispersed in the continuous phase; and
c) a dual emulsifier system consisting essentially of an organosilicone hydrophobic surfactant and an organosilicone hydrophilic surfactant.

2. The aqueous emulsion of claim 1, wherein the liquid alkoxytrimethylsilane is present in the emulsion in an amount ranging from about 1 to about 50 weight percent.

3. The aqueous emulsion of claim 2, wherein the liquid alkoxytrimethylsilane is present in the discontinuous phase of the emulsion in an amount ranging from about 1 to 100 weight percent.

4. The aqueous emulsion of claim 1, wherein the liquid alkoxytrimethylsilane has the general formula ROSiR′₃, wherein R is an aliphatic hydrocarbon substituent with from 12 to about 22 carbon atoms, which may have acyclic, cyclic or both structures, and which may contain single or multiple unsaturations, and R′ is an alkyl group of 1 to 6 carbon atoms.

5. The aqueous emulsion of claim 4, wherein R is a stearyl group.

6. The aqueous emulsion of claim 4 wherein R is a retinyl group.

7. The aqueous emulsion of claim 4 wherein the alkoxytrimethylsilane is stearoxytrimethylsilane.

8. The aqueous emulsion of claim 4, wherein the alkoxytrimethylsilane is retinoxytrimethylsilane.

9. The aqueous emulsion of claim 1, wherein the organosilicone hydrophobic surfactant is of an Alkyl PEG/PPG-x/y dimethicone copolymer or an Alkyl PEG/PPG-x/y methicone polymer, or an Alkyl bis(PEG/PPG-x/y) dimethicone polymer, wherein Alkyl is a saturated hydrocarbon substituent with from 6 to about 18 carbons, PEG is polyoxyethylene, PPG is polyoxypropylene, and x and y may independently range from 1 to about 20, and wherein said organosilicone hydrophobic surfactant is present in the emulsion at a concentration ranging from about one to about three molecules per 100 square Angstroms of surface area of the alkoxytrimethylsilane particles.

10. The aqueous emulsion of claim 1, wherein the organosilicone hydrophilic surfactant comprises a water soluble Trisiloxane PEG-x copolymer, wherein the Trisiloxane is (Me₃SiO)₂MeSi—, Me is methyl, PEG is polyoxyethylene, and x may range from 1 to about 20, and wherein said organosilicone hydrophilic surfactant is present in the emulsion at a concentration of from two to about six molecules per 100 square Angstroms of surface area of the organosilicone hydrophobic surfactant treated particles.

11. The aqueous emulsion of claim 9, wherein the organosilicone hydrophobic surfactant comprises a Caprylyl PEG/PPG-20/20 dimethicone copolymer.

12. The aqueous emulsion of claim 10, wherein the organosilicone hydrophilic surfactant comprises a Trisiloxane PEG-10 copolymer.

13. The aqueous emulsion of claim 1, wherein the discontinuous phase is substantially comprises of particles having a diameter of less than about 500 nanometers.

14. The aqueous emulsion of claim 1, further comprising cosmetic components selected from the group consisting of a buffer, a biocide, a foaming agent, an anti-foaming agent, a fragrance, a colorant, a thickening agent and mixtures of any of the foregoing.

15. The aqueous emulsion of claim 1 wherein said discontinuous phase further comprises an inert organic diluent for the alkoxytrimethylsilane.

16. A cosmetic formulation for treatment of the skin or hair wherein the composition comprises an aqueous emulsion comprising:
a) a continuous phase comprising water; and
b) a discontinuous phase comprising at least one liquid alkoxytrimethylsilane, wherein the discontinuous phase forms particles which are uniformly dispersed in the continuous phase; and
c) a dual emulsifier system consisting essentially of an organosilicone hydrophobic surfactant and an organosilicone hydrophilic surfactant.

17. The cosmetic formulation of claim 16, wherein the liquid alkoxytrimethylsilane is present in the emulsion in an amount ranging from about 1 to about 50 weight percent.

18. The cosmetic formulation of claim 17, wherein the liquid alkoxytrimethylsilane is present in the discontinuous phase of the emulsion in an amount ranging from about 1 to about 100 weight percent.

19. The cosmetic formulation of claim 16, wherein the liquid alkoxytrimethylsilane has the general formula ROSiR′₃, wherein R is an aliphatic hydrocarbon substituent with from 12 to about 22 carbon atoms, which may have acyclic, cyclic or both structures, and which may contain single or multiple unsaturations, and R′ is an alkyl group of 1 to 6 carbon atoms.

20. The cosmetic formulation of claim 19, wherein R is a stearyl group.

21. The cosmetic formulation of claim 19 wherein R is a retinyl group.

22. The cosmetic formulation of claim 19 wherein the alkoxytrimethylsilane is stearoxytrimethylsilane.

23. The cosmetic formulation of claim 19, wherein the alkoxytrimethylsilane is retinoxytrimethylsilane.

24. The cosmetic formulation of claim 16, wherein the organosilicone hydrophobic surfactant comprises an Alkyl PEG/PPG-x/y dimethicone copolymer or an Alkyl PEG/PPG-x/y methicone polymer, or an Alkyl bis(PEG/PPG-x/y) dimethicone polymer, wherein Alkyl is a saturated hydrocarbon substituent with from 6 to about 18 carbons, PEG is polyoxyethylene, PPG is polyoxypropylene, and x and y may independently range from 1 to about 20, and wherein said organosilane hydrophobic surfactant is present in the emulsion at a concentration of from one to about three molecules per 100 square Angstroms of surface area of the alkoxytrimethylsilane particles.

25. The cosmetic formulation of claim 16, wherein the organosilicone hydrophilic surfactant comprises a water soluble Trisiloxane PEG-x copolymer, wherein the Trisiloxane is (Me₃SiO)₂MeSi—, Me is methyl, PEG is polyoxyethylene, and x may range from 1 to about 20, and wherein said organosilane hydrophilic surfactant is present in the emulsion at a concentration of from two to about six molecules per 100 square Angstroms of surface area of the organosilicone hydrophobic surfactant treated particles.

26. The cosmetic formulation of claim 24, wherein the organosilicone hydrophobic surfactant comprises a Caprylyl PEG/PPG-20/20 dimethicone copolymer.

27. The cosmetic formulation of claim 25, wherein the organosilicone hydrophilic surfactant comprises a Trisiloxane PEG-10 copolymer.

28. The cosmetic formulation of claim 16, wherein the discontinuous phase is substantially comprised of particles having a diameter of less than about 500 nanometers.

29. The cosmetic formulation of claim 16, further comprising cosmetic components selected from the group consisting of a buffer, a biocide, a foaming agent, an anti-foaming agent, a fragrance, a colorant, a thickening agent and mixtures of any of the foregoing.

30. The cosmetic formulation of claim 16 wherein said discontinuous phase further comprises an inert organic diluent for the alkoxytrimethylsilane.
